(19) United States
(12) Patent Application Publication
(10) Pub. No.: US 2009/0099354 A1
(43) Pub. Date: Apr. 16, 2009

(54) METHOD FOR SEPARATING POLYMERIC PENTOSE FROM A LIQUID/SLURRY

(75) Inventors: Peter Axegard, Solna (SE); Karin Lindgren, Stockholm (SE); Olof Dahlman, Stockholm (SE); Per Tomani, Huddinge (SE); Fredrik Lundqvist, Ekero (SE)

Correspondence Address: YOUNG & THOMPSON, 209 Madison Street, Suite 500, ALEXANDRIA, VA, 22314, US

(73) Assignee: STFI-PACKFORSK AB, STOCKHOLM (SE)

(21) Appl. No.: 12/293,090

(22) PCT Filed: Feb. 2, 2007
(86) PCT No.: PCT/SE2007/000095

§ 371 Date: Oct. 21, 2008

Related U.S. Application Data
(60) Provisional application No. 60/782,524, filed on Mar. 16, 2006.

(30) Foreign Application Priority Data
Mar. 16, 2006 (SE) 0600583-9

(51) Int. Cl. C07H 1/00

(52) U.S. Cl. 536/128; 536/127

(57) ABSTRACT

The present invention provides a method for extracting polymeric pentose, preferably xylan, from a polymeric pentose containing liquid/slurry, preferably black liquor, comprising the following steps: a) providing a polymeric pentose containing liquid/slurry; b) concentrating the polymeric pentose by membrane separation of a said polymeric pentose containing liquid/slurry; and c) adding alcohol to the concentrate obtained in step b) and subsequently acidifying said concentrate obtained in step b) for precipitating polymeric pentose whereby a polymeric pentose product, or an intermediate polymeric pentose product, is obtained. In this above way polymeric pentose, e.g. xylan, is separated whereby the use of methanol (less lignin must be kept in solution) and acid (less acid which buffers the pH-decrease) become much lower. The present invention also provides according to a second aspect a polymeric pentose product or an intermediate polymeric pentose product obtainable by the method according to the first aspect. The present invention also provides according to a third aspect use, preferably for the production of food additives or as chemical feed stock, of the polymeric pentose product or the intermediate polymeric pentose product of the second aspect of the invention.

This invention concerns the technical field of polymeric pentose separation, preferably xylan separation. In particular the present invention relates to a method for polymeric pentose separation from a polymeric pentose containing liquid/slurry, such as process liquors in a mill containing polymeric pentose, preferably black liquor.

Further the invention relates to polymeric pentose products obtainable by the above mentioned method and use of said products.

### BACKGROUND

Black liquor contains significant amounts of valuable polymeric pentose compounds, e.g. xylan, and other high-molecular carbohydrates. There is presently no technical method with a reasonable economy which can separate these from the high-molecular lignin. The only way presently to use these compounds are through burning them in the so called soda recovery unit where the energy content is extracted in the form of steam of high quality. Methods which in a technical and economical way can be used for separating these high-molecular carbohydrates and lignin respectively would be desirable as the organic components are more valuable in pure form than their energy content.

Membrane separation of black liquor gives a mixture of high-molecular carbohydrates and lignin, but needs a further separation step. A possible solution is to add acid to the concentrate to pH 7-9 which gives a precipitation of lignin whereas xylan remains in the solution.

Addition of methanol to black liquor (two parts methanol to one part of black liquor) followed by acidifying to pH 7 gives a on the other hand a precipitation of xylan and other hemicellulosic components whereas the lignin remains in the methanol enriched solution. The methanol consumption is however very high for this to be feasible economically. In both cases also the high acid consumption brings about high direct and indirect costs.

Through CN 1687094 a method for purifying xylan by using addition of ethanol and subsequently phoshoric acid or sulfuric acid is disclosed. However nothing is mentioned about membrane separation of the raw material before the addition of ethanol.

It is accordingly a problem, however, with the high consumption of alcohol and acid when separating polymeric pentose compounds, e.g. xylan, from black liquor. It would thus be desirable to have a method for separating polymeric pentose compounds from a polymeric pentose containing liquid/slurry whereby the separation is economical, e.g. minimal amounts of alcohol and/or acid may be applied for the separation of polymeric pentose, e.g. xylan.

### SUMMARY OF THE INVENTION

The present invention solves one or more of the above problems, whereby both of the above methods are combined, by providing according to a first aspect a method for extracting polymeric pentose, preferably xylan, from a polymeric pentose containing liquid/slurry polymeric pentose containing liquid/slurry, preferably black liquor, comprising the following steps:
a) providing a polymeric pentose containing liquid/slurry;
b) concentrating the polymeric pentose by membrane separation of a said polymeric pentose containing liquid/slurry; and
c) adding alcohol to the concentrate obtained in step b) and subsequently acidifying said concentrate obtained in step b) for precipitating polymeric pentose whereby a polymeric pentose product, or an intermediate polymeric pentose product, is obtained.
   In this above way the polymeric pentose product, preferably xylan, is separated whereby the use of alcohol, e.g. methanol (less lignin must be kept in solution), and acid (less acid which buffers the pH-decrease) become much lower.
   The present invention also provides according to a second aspect a polymeric pentose (preferably xylan) product or an intermediate polymeric pentose (preferably xylan) product obtainable by the method according to the first aspect. The present invention also provides according to a third aspect use, preferably for the production of food additives or as chemical feed stock, of the polymeric pentose product or the intermediate polymeric pentose product of the second aspect.

### DETAILED DESCRIPTION OF THE INVENTION

It is intended throughout the present description that the expression “polymeric pentose containing liquid/slurry” is any liquid or slurry, which contains polymeric pentose compounds, e.g. xylan. This liquid or slurry may be a process liquor, containing e.g. xylan, in a mill, preferably said liquid or slurry is a black liquor.

It is intended throughout the present description that the expression “compound comprising sulphate or a sulphate ion” embraces any compound comprising sulphate or a sulphate ion. This compound may be Na—, K—, (Al—), Ca—, Mg—, Fe— or organic sulfate, CaSO₄, K₂SO₄, Al₂SO₄, iron sulfates or MgSO₄. Said compound may also be comprised in recovery boiler ashes, which is a mixture, or it may be essentially pure Na₂SO₄.

It is intended throughout the present description that the expression “acidifying” embraces any means for acidifying the liquid/slurry, such as black liquor. Preferably the acidifying is performed by adding SO₂(g), organic acids, HCl, HNO₃, carbon dioxide or sulphuric acid (in the form of fresh sulfuric acid or a so called “spent acid” from a chlorine dioxide generator) or mixtures thereof to said liquid/slurry (preferably black liquor) most preferred by adding carbon dioxide or sulphuric acid. Especially preferred is the use of carbon dioxide when acidifying in step c) above.

It is intended throughout the present description that the expression “dewatering” embraces any means for dewatering. Preferably the dewatering is performed by using centrifugation, a filter press apparatus, a band filter, a rotary filter, such as a drum filter, or a sedimentation tank, or similar equipment, most preferred a filter press apparatus is used.

According to a preferred embodiment of the first aspect of the invention the alcohol in step c) is methanol, ethanol, or a mixture thereof, preferably methanol.

According to a preferred embodiment of the first aspect of the invention the membrane separation of step b) is achieved by using ultrafiltration, preferably by using a membrane with a cut off of from 1000 to 50000 Da.

According to a preferred embodiment of the first aspect of the invention the polymeric pentose containing liquid/slurry is a black liquor provided after alkaline delignification, preferably a black liquor resulting from the initial phase of the cooking. If then separating xylan, the xylan is then less decomposed and at the same time the lignin concentration is relatively low.

According to a preferred embodiment of the first aspect of the invention the membrane separation of step b) is performed at present temperature, alkalinity and pressure.

According to a preferred embodiment of the first aspect of the invention the alcohol addition of step c) is performed at overpressure, whereupon the alcohol is removed through pressure decrease and is then recycled back for use in said step c).

According to a preferred embodiment of the first aspect of the invention the membrane separation of step b) is performed at cooking temperature whereupon the pressure in the concentrate is adjusted to a level facilitating simple regeneration of alcohol for use in said step c).

According to a preferred embodiment of the first aspect of the invention the alcohol addition of step c) is performed using virgin methanol and/or methanol obtained from methanol containing non-condensable gases.

According to a preferred embodiment of the first aspect of the invention the polymeric pentose containing liquid/slurry emanates from hardwood, softwood, annual plants or a combination thereof.

According to a preferred embodiment of the first aspect of the invention the membrane separation of step b) is performed at full cooking temperature, cook alkalinity and with a pressure facilitating simple recycling of the remaining liquor to the cooking liquor, when lignin depleting treatment is not comprised in said method.

According to a preferred embodiment of the first aspect of the invention the polymeric pentose containing liquid/slurry, preferably a black liquor, is pretreated, or treated between step b) and c), for depleting lignin using a method comprising the following steps:
i) addition of one or more compounds comprising sulphate or sulphate ions, or a mixture comprising said compound, to said liquid/slurry;
ii) adjustment of the pH level of the said liquid/slurry by acidifying, and
iii) separating of a lignin product, or an intermediate lignin product.

According to a preferred embodiment of the first aspect of the invention the separating of step iii) is performed in a filter press apparatus.

According to a preferred embodiment of the first aspect of the invention the addition of step i) is done by adding recovery boiler ashes, i.e. ashes emanating from a soda recovery unit, which is a steam generator combined with a smelting furnace for the utilization of the heat of combustion of the black liquor and the recovery of the greater part of its inorganic components, or Na₂SO₄, CaSO₄, K₂SO₄, Al₂SO₄, iron sulfates or MgSO₄. Preferably Na₂SO₄ is used.

According to a preferred embodiment of the first aspect of the invention the mixing is performed after the adjustment of the pH level in step ii).

According to a preferred embodiment of the first aspect of the invention the pH level is adjusted to below approximately pH 11 in step ii), preferably in the range of from pH 8 to pH 11.

According to a preferred embodiment of the first aspect of the invention the pH level is adjusted whereby using CO₂.

According to a preferred embodiment of the first aspect of the invention the temperature is varied from 20 to 200° C. depending on the nature of said liquid/slurry.

According to a preferred embodiment of the first aspect of the invention the polymeric pentose containing liquid/slurry, preferably a black liquor, is pretreated, or treated between step b) and c), for depleting lignin using a method comprising the following step:
iv) precipitating of lignin by acidifying said liquid/slurry and thereupon separation of lignin, and optionally the following steps
v) suspending the lignin filter cake obtained in step iv) whereupon a second suspension is obtained and adjusting the pH level to approximately the pH level of the washing water,
vi) dewatering of the second suspension,
vii) addition of washing water and performing a displacement washing at more or less constant conditions without any dramatic gradients in the pH, and
viii) dewatering of the filter cake produced in step vii) into a high dryness and displacement of the remaining washing liquid in said filter cake, whereby a lignin product or an intermediate lignin product is obtained.

According to a preferred embodiment of the first aspect of the invention the separation of step iv) and/or the dewatering of step vi) and/or viii) is performed in a filter press apparatus where the filter cake is blown through by gas or a mixture of gases, preferably flue gases, air or vapor, most preferred air or overheated vapor, in order to dispose of the remaining liquid/slurry.

According to a preferred embodiment of the first aspect of the invention the pH level is adjusted to below approximately pH 11 in step v), preferably in the range of from pH 8 to pH 11.

According to a preferred embodiment of the first aspect of the invention the washing water has a pH level of below approximately pH 11, preferably in the range of from pH 8 to pH 11.

According to a preferred embodiment of the first aspect of the invention the filter cake obtained in step iv) is blown through by using gas or a mixture of gases, including e.g. flue gases, air and vapor, which preferably can be air or overheated vapor, before suspending said cake as set out in step v).

According to a preferred embodiment of the first aspect of the invention the pH level adjustment is combined with an adjustment of the ion strength, preferably by using multivalent alkaline earth metal ions, most preferred calcium ions.

According to a preferred embodiment of the first aspect of the invention the pH level adjustment is combined with an adjustment of the ion strength corresponds to the pH level and ion strength of the washing liquid.

In this preferred embodiment the lignin is stabilized during the washing, as set out above earlier, whereby a pH-decrease is combined with an adjustment of the ionic strength in the slurry stage, preferably with multivalent alkaline earth metal ions (e.g. calcium ions). At a given pH, a higher ionic strength in the suspension stage reduces the lignin yield losses. Here also the ionic strength and pH of the wash water essentially corresponds to the conditions in the slurry stage to avoid gradients during the washing process. A higher ionic strength in the slurry and in the wash water gives a stable lignin even at high pH-values. Besides making the washing easier, divalent calcium ions can be introduced into the lignin, which in the combustion of the lignin can bind sulfur in the form of calcium sulphate (Aarsrud et al 1990, WO 9006964).

According to a preferred embodiment of the first aspect of the invention the remaining washing liquor in the filter cake in step viii) is removed with air or flue gases, preferably flue gases from a recovery boiler, a lime kiln or a bark boiler.

According to a preferred embodiment of the first aspect of the invention the washing liquor and a part of the filtrate from the second dewatering in step viii) is returned to the re-slurrying stage step v) to further reduce the consumption of acid and water.

Preferred features of each aspect of the invention are as for each of the other aspects mutatis mutandis. The prior art documents mentioned herein are incorporated to the fullest extent permitted by law. The invention is further described in the following examples, together with the appended figures, which do not limit the scope of the invention in any way. Embodiments of the present invention are described in more detail with the aid of examples of embodiments, together with the appended figures, the only purpose of which is to illustrate the invention and are in no way intended to limit its extent.

### FIGURES

FIG. 1 shows the molar mass distribution diagram for the glucuronoxylan isolated from birch black liquor in example 2 below.

FIG. 2 shows the molar mass distribution diagram for the glucuronoxylan isolated from eucalyptus black liquor in example 3 below.

### EXAMPLES

### Example 1

When exploiting the observation that in the initial phase of the cooking there is primarily a releasing of xylan in the black liquor whereas the lignin releasing is minimal, it is then at that point possible to apply a membrane filtration (cut off 3000 Da) leaving the xylan in the retentate and the lignin in the filtrate. After that separation, methanol is added followed by an acidification to about pH 9, preferably through the addition of CO₂. This enables a maximum separation of xylan whereby using a minimum amount of methanol and acid. Compared with separating xylan from black liquor after the cooking, the methanol consumption is reduced by approximately 90%, with maintained separation of xylan. Also the acid addition is reduced.

In comparison with the method described in CN 1687094, the method according to the first aspect of the present invention is operable at a pH range which is above the one specified in said document which means that a less amount of acid is necessary in the method according to the first aspect of the present invention in comparison with the method described in CN 1687094. In addition, the amount of alcohol needed in the method according to the first aspect of the present invention is clearly lower than the amount of alcohol necessary in the method described in CN 1687094. In CN 1687094 the amount of ethanol is 1-5 times the volume of extract whereas the amount of ethanol needed in the method according to the first aspect of the present invention is about 1/10 to 2/10 of the volume of the extract (black liquor). Thus the amount of alcohol can be lowered with at least 2/3 when using the method according to the first aspect of the present invention in comparison with the method set out in CN 1687094. Also the product according to the second aspect differs from the product of CN 1687094 because the product according to the second aspect contains a less amount of low molecular xylan due to the use of the method according to the first aspect of the present invention. Accordingly the method according to the first aspect of the present invention does not only provide a more efficient method for extracting polymeric pentose, but also a better product in comparison with the method set out in CN 1687094.

### Example 2

### Separation of Glucuronoxylan From Birch Kraft Black Liquor.

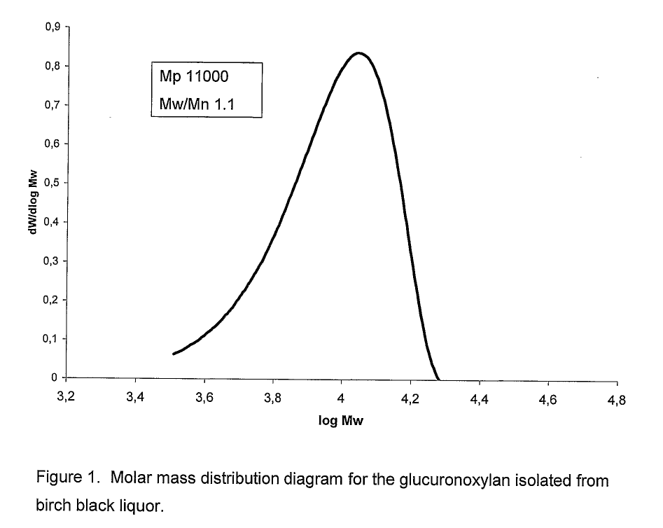

A black liquor containing 12 g/L xylan and 7 g/L lignin was recovered from the initial phase in kraft cooking of birch wood chips (Effective alkali=18%, Sulphidity=38%, H Factor=5). This black liquor was membrane separated by ultrafiltration at temperature 120° C. employing a ceramic membrane with a nominal cut off 5000 Da (trans membrane pressure 3.5 bar). The xylan enriched ultrafiltration retentate thus obtained contained 31 g/L xylan and 10 g/L lignin. Two volumes of methanol was added to one volume of the birch black liquor ultrafiltration retentate and the pH of the resulting mixture was adjusted to ˜9 by addition of concentrated sulphuric acid. The voluminous precipitate thus formed was recovered by centrifugation, subsequently washed with methanol/water and finally dried. The yield of the birch glucuronoxylan precipitate was 4% on wood basis. The chemical composition and purity of the birch xylan product isolated is reported in Table 1. The molar mass distribution for this product, as determined by aqueous Size-Exclusion Chromatography (SEC), is described in FIG. 1.
The abbreviations appearing in the figure have the following meanings:
Mp=Peak molecular mass (i.e. molecular mass at SEC top maximum)
Mn=Number average molecular mass (calculated through use of the formula appearing below)
Mw=Weight average molecular mass (calculated through use of the formula appearing below)

$$\overline{{M}_{n}} = \frac{{\sum }_{i} {n}_{i} {M}_{i}}{{\sum }_{i} {n}_{i}} = \frac{{\sum }_{i} {A}_{i}}{{\sum }_{i} {A}_{i} / {M}_{i}}$$

$$\overline{{M}_{w}} = \frac{{\sum }_{i} {n}_{i} {M}_{i}^{2}}{{\sum }_{i} {n}_{i} {M}_{i}} = \frac{{\sum }_{i} {A}_{i} {M}_{i}}{{\sum }_{i} {A}_{i}}$$

### Example 3

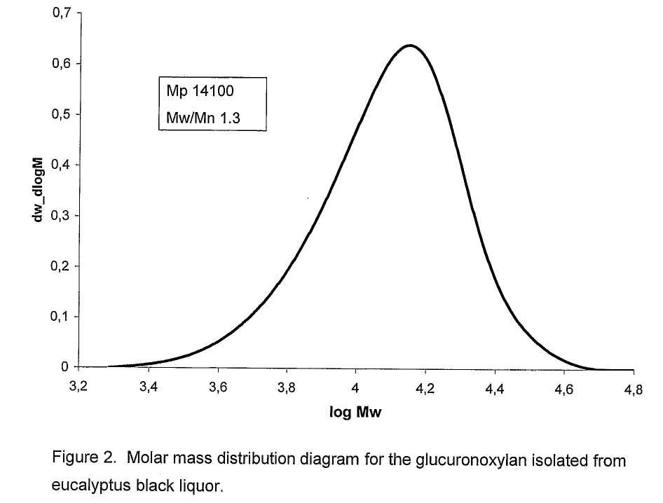

Separation of Glucuronoxylan from Eucalyptus Kraft Black Liquor.
A black liquor containing 5 g/L xylan and 6 g/L lignin was recovered from the initial phase in kraft cooking of eucalyptus wood chips (Effective Alkali=17%, Sulphidity=38%, H Factor=70). This black liquor was membrane separated by ultrafiltration at a temperature 120° C. employing a ceramic membrane with a nominal cut off 5000 Da (trans membrane pressure 3.5 bar). The xylan enriched ultrafiltration retentate thus obtained contained 15 g/L xylan and 11 g/L lignin. Two volumes of methanol was added to one volume of the eucalyptus black liquor ultrafiltration retentate and the pH of the resulting mixture was adjusted to ˜9 by addition of sulphuric acid. The voluminous precipitate thus formed was recovered by centrifugation, subsequently washed with methanol/water and finally dried. The yield of the eucalyptus glucuronoxylan precipitate was 1.5% on wood basis. The chemical composition and purity of the eucalyptus xylan product isolated is reported in Table 1. The molar mass distribution of this product, as determined by aqueous Size-Exclusion Chromatography (SEC), is described in FIG. 2. The abbreviations appearing in the figure have the same meanings as set out under example 2 above.

TABLE 1
Chemical composition and purity of the birch and eucalyptus
glucuronoxylans separated from black liquors.

| | | |
|---|---|---|
| Glucuronoxylan from black liquor | Birch | Eucalyptus |

Composition (weight-%)

| | | |
|---|---|---|
| Carbohydrates | 92.7 | 93.0 |
| Lignin | 7.3 | 7.0 |

Sugar composition (weight-%)

| | | |
|---|---|---|
| Xylose | 91.6 | 82.9 |
| 4-O-Methyl-glucuronic acid | 6.1 | 5.5 |
| Hexenuronic acid | 1.9 | 7.9 |
| Galactose | 0.4 | 2.2 |
| Glucose | 0.0 | 1.1 |
| Arabinose | 0.0 | 0.4 |
| Mannose | 0.0 | 0.0 |
| Galacturonic acid | 0.0 | 0.0 |
| Glucuronic acid | 0.0 | 0.0 |
| Degree of substitution, Ds, mol-% | 6.4 | 13.5 |
| Ds of 4-O-Methyl-glucuronic acid, mol-% | 4.7 | 5.6 |
| Ds of Hexenuronic acid, mol-% | 1.7 | 7.9 |

Various embodiments of the present invention have been described above but a person skilled in the art realizes further minor alterations, which would fall into the scope of the present invention. The breadth and scope of the present invention should not be limited by any of the above-described exemplary embodiments, but should be defined only in accordance with the following claims and their equivalents. For example, any of the above-noted methods can be combined with other known methods e.g. for separating xylan from a xylan containing liquid/slurry, such as black liquor. Other aspects, advantages and modifications within the scope of the invention will be apparent to those skilled in the art to which the invention pertains.

### LIST OF DOCUMENTS APPEARING IN THE DESCRIPTION

CN 1687094, and

WO 9006964

1. A method for extracting polymeric pentose, preferably xylan, from a polymeric pentose containing liquid/slurry comprising the following steps: a) providing a polymeric pentose containing liquid/slurry; b) concentrating the polymeric pentose by membrane separation of a said polymeric pentose containing liquid/slurry; and c) adding alcohol to the concentrate obtained in step b) and subsequently acidifying said concentrate obtained in step b) for precipitating polymeric pentose whereby a polymeric pentose product, or an intermediate polymeric pentose product, is obtained.

2. A method according to claim 1 wherein the polymeric pentose containing liquid/slurry is black liquor.

3. A method according to claim 1 wherein the alcohol in step c) is methanol, ethanol, or a mixture thereof, preferably methanol.

4. A method according to claim 1 wherein the membrane separation of step b) is achieved by using ultrafiltration, preferably by using a membrane with a cut off of from 1000 to 50000 Da.

5. A method according to claim 1 wherein the polymeric pentose containing liquid/slurry is a black liquor provided after alkaline delignification, preferably a black liquor resulting from the initial phase of the cooking.

6. A method according to claim 1 wherein the membrane separation of step b) is performed at present temperature, alkalinity and pressure.

7. A method according to claim 1 wherein the alcohol addition of step c) is performed at overpressure, whereupon the alcohol is removed through pressure decrease and is then recycled back for use in said step c).

8. A method according to claim 2 wherein the membrane separation of step b) is performed at cooking temperature whereupon the pressure in the concentrate is
adjusted to a level facilitating simple regeneration of alcohol for use in said step c).

9. A method according to claim 3 wherein the alcohol addition of step c) is performed using virgin methanol and/or methanol obtained from methanol containing non-condensable gases.

10. A method according to claim 1 wherein the polymeric pentose containing liquid/slurry emanates from hardwood, softwood, annual plants or a combination thereof.

11. A method according to claim 2 wherein the membrane separation of step b) is performed at full cooking temperature, cook alkalinity and with a pressure facilitating simple recycling of the remaining liquor to the cooking liquor, when lignin depleting treatment is not comprised in said method.

12. A method according to claim 1 wherein the polymeric pentose containing liquid/slurry, preferably a black liquor, is pretreated, or treated between step b) and c), for depleting lignin using a method comprising the following steps: i) addition of one or more compounds comprising sulphate or sulphate ions, or a mixture comprising said compound, to said liquid/slurry; ii) adjustment of the pH level of the said liquid/slurry by acidifying, and iii) separating of a lignin product, or an intermediate lignin product.

13. A method according to claim 12 wherein the separating of step iii) is performed in a filter press apparatus.

14. A method according to claim 12 wherein the addition of step i) is done by adding recovery boiler ashes or Na₂Sθ4, CaSO₄, K₂SO₄, AI₂SO₄, iron sulfates and/or MgSO₄, preferably Na₂Sθ4.

15. A method according to claim 12 wherein mixing is performed after the adjustment of the pH level in step ii).

16. A method according to claim 12 wherein the pH level is adjusted to below
approximately pH 11 in step ii), preferably in the range of from pH 8 to pH 11.

17. A method according to claim 12 wherein the pH level is adjusted whereby using CO₂.

18. A method according to claim 12 wherein the temperature is varied from 20 to 200° C. depending on the nature of said liquid/slurry.

19. A method according to claim 1 wherein the polymeric pentose containing liquid/slurry, preferably a black liquor, is pretreated, or treated between step b) and c), for depleting lignin using a method comprising the following step: iv) precipitating of lignin by acidifying said liquid/slurry and thereupon separation of lignin, and optionally the following steps:
v) suspending the lignin filter cake obtained in step iv) whereupon a second suspension is obtained and adjusting the pH level to approximately the pH level of the washing water, vi) dewatering of the second suspension, vii) addition of washing water and performing a displacement washing at more or less constant conditions without any dramatic gradients in the pH, and viii) dewatering of the filter cake produced in step vii) into a high dryness and displacement of the remaining washing liquid in said filter cake, whereby a lignin product or an intermediate lignin product is obtained.

20. A method according to claim 19 wherein the separation of step iv) and/or the dewatering of step vi) and/or viii) is performed in a filter press apparatus where the filter cake is blown through by gas or a mixture of gases, preferably flue gases, air or vapor, most preferred air or overheated vapor, in order to dispose of the remaining liquid/slurry.

21. A method according to claim 19 wherein the pH level is adjusted to below approximately pH 11 in step v), preferably in the range of from pH 8 to pH 11.

22. A method according to claim 19 wherein the washing water has a pH level of below approximately pH 11, preferably in the range of from pH 8 to pH 11.

23. A method according to claim 19 wherein the filter cake obtained in step iv) is blown through by using gas or a mixture of gases, including e.g. flue gases, air and vapor, which preferably can be air or overheated vapor, before suspending said cake as set out in step v).

24. A method according to claim 19 wherein the pH level adjustment is combined with an adjustment of the ion strength, preferably by using multivalent alkaline earth metal ions, most preferred calcium ions.

25. A method according to claim 19 wherein the pH level adjustment is combined with an adjustment of the ion strength corresponds to the pH level and ion strength of the washing liquid.

26. A method according to claim 19 wherein the remaining washing liquor in the filter cake in step viii) is removed with air or flue gases, preferably flue gases from a recovery boiler, a lime kiln or a bark boiler.

27. A method according to claim 19 wherein the washing liquor and a part of the filtrate from the second dewatering in step viii) is returned to the re-slurrying stage step v) to further reduce the consumption of acid and water.

28. A polymeric pentose product or an intermediate polymeric pentose product obtainable by a method according to claim 1.

29. (canceled)
